(19) United States
(12) Patent Application Publication
(10) Pub. No.: US 2017/0049766 A1
(43) Pub. Date: Feb. 23, 2017

(54) METHOD AND DOSE FOR THE ADMINISTRATION OF DEXMEDETOMIDINE, FENTANYL AND PROPOFOL FOR CONSCIOUS SEDATION IN SURGERY

(71) Applicant: DOCTOR RODRIGO TORRES CIAPPA Y CIA LTDA., Santiago, Región Metropolitana (CL)

(72) Inventor: CARLOS RODRIGO TORRES CIAPPA, Santiago (CL)

(21) Appl. No.: 15/107,911

(22) PCT Filed: Dec. 23, 2014
(86) PCT No.: PCT/CL2014/000085

§ 371 Date: Oct. 7, 2016

(30) Foreign Application Priority Data
Dec. 30, 2013 (CL) 2013-3803

(51) Int. Cl. A61K 31/4468; A61K 31/05; A61K 31/167; A61K 31/4174

(52) U.S. Cl. CPC A61K 31/4468; A61K 31/4174; A61K 31/05; A61K 31/167

(57) ABSTRACT

The invention refers to a method and doses in the administration of drugs for conscious sedation of a patient to be used in surgeries, including spinal column endoscopic surgery and other surgeries, as well as other applications inside or outside the operating theater using bio-backfeed or communication with the patient him/herself. Conscious sedation in surgeries, including spine or similar or other surgeries and applications inside and outside the operating theater permit the surgeon a greater risk control, e.g. injure the patient column or other organs or parts of the patient who, when being watchful there is communication with the patient, but it does not occur at present in operations involving general anesthesia; the latter includes column or similar surgeries, other surgeries and applications inside and outside the operating theater.

### DESCRIPTION OF THE PRIOR ART

Only a few centers exist where minimum invasive surgery and column endoscopic surgery are performed in the world; the elected anesthesia technique applied in them is general anesthesia or some type of sedation for implementing the procedures.

The customary election according to the traditional survey technique is general anesthesia. This type of survey is widely used all over the world, being applied through ample surgical incisions in order to gain access to the site of the lesion is always under general anesthesia.

Bearing in mind the technique status, we can cite U.S. Pat. No. 7,247,154, which refers to a method for sedating a patient, as compared with the patient being submitted to one or more medical and/or surgical procedures without general anesthesia; said method comprises:

The step of connecting a patient to a specific capable to administer control drugs to the patient during the procedure; said specific supply device is coupled to a controller of specific to the patient.

Connect at least one device with a patient health monitor, wherein said monitor generates a signal that reflects at least to monitor the physiological condition of the patient during the procedure.

Coupling at least one device with a health monitor for patients to a patient; said health monitor device generates a signal that reflects at least one monitoring step of the patient physiological condition during the procedure.

Delivery of one or more drugs to the patient in the course of the procedure, using the drug supply device, which delivers same at the desired supply speed.

Sending said signal to the monitoring condition device to the controller during the procedure; the communication signal of a current value reflects the supervised physiological condition of the patient.

The access parameters for said monitoring of the physiological conditions; said parameters indicate the values of the monitoring measurements of the physiological condition, where said values are correlated with the desired sedation levels in the course of said procedure;

A comparison of the measurements in the received signal reflects said parameters, and

Modifying the supply speed of the drugs as necessary in response to said comparison, in order to safely maintain the patient within the desired sedation levels throughout the procedure.

In what concerns the status of technique, we can cite the invention U.S. Pat. No. 7,247,154 B2, which refers to a method for sedating a patient submitted to one or more medical and/or surgical procedures without general anesthesia; said method comprises the connection of a device to the patient; the supply of a controlled drug during said procedure, wherein the drug supplying device is coupled to a controller, which controls the supply of drugs to the patient; couple at least a device being capable to monitor the patient health. Said device should be capable to generate a signal that reflects at least one monitoring step of the patient physiological condition during the procedure; delivery of one or more drugs to the patient during said procedure using the drugs supplying device; the specific is administered at the convenient supply speed and the signal is sent from the health monitoring device to the controller throughout the procedure. The value of said communication signal in real time reflects the monitored physiological condition of the patient; access to the physiological condition parameters through said monitoring, wherein the parameters indicate the values of the monitoring measurements of the physiological condition in which said values are correlated to the desired sedation levels in the course of the procedure; the comparison of said measurements, which are reflected in a signal received from said parameters and to modify the drugs desired supply speed as necessary in response to said comparison, with a view to maintaining the patient in a safe condition within said desired sedation levels throughout the procedure.

Another invention Patent—U.S. Pat. No. 2,888,922 (Bellville) concerns a servo-controlled drugs administration device for the automatic and continuous non-conscious patient, based on a voltage being representative of the patient cortical activity obtained by means of an electroencephalogram (EEG). The device continues and automatically and robotically increases or decreases the anesthetic gas flow (or IV infusion) in response to selected frequencies of the brain potential for maintaining a constant unconsciousness level.

### DESCRIPTION OF THE INVENTION AND FIELD OF APPLICATION

The invention applying for a patent concerns a method and dose in the administration of drugs or sedation to a conscious patient, based on specific needs using backfeed in communication with the same patient for use in surgeries, including endoscopic surgery of spinal column and applications inside or outside the operating theater. When a conscious patient is involved, this permits the reduction of risks and/or increased safety. Sedation of conscious patient in surgeries, including spine endoscopic surgeries and other applications and surgeries inside and outside the operating theater permits the surgeon greater control and avoids injuring the patient, including the spine or other organs or parts of the body, which an alert condition provides communication with the patient; this is not the case at present for operations including general anesthesia, which comprise spine surgeries or similar and in other operations and applications inside or outside the operating theater. A participative, conscious, safe and flexible sedation method is proposed (“FACIVS”) for minimal invasive surgery, including spine and other surgeries inside and outside the operating theater.

Using the concept of multimodal analgesia, the proposed sedation is based on an idea of multimodal anesthesia through the administration of specifics acting at different levels of the central nervous system. This method generates a sedate type that permits the active participation of the patient during the whole surgical process he/she is undergoing. This sedation type brings about clinical and safety benefits that should promote its future election In a minimal invasive endoscopic techniques of the spinal column.
Low doses of associated drugs are used (dexmedetomidine, phentanil and propophol) and some lidocaine or other short action opiate.
Fast and completion of the sedation with scant residual effects.
Gradual and foreseeable hemodynamic changes in patients whose surgical position is not modified.
The surgical position should be comfortable. Undesired compression on body parts is avoided, because the patient always alerts the surgeon of this situation.
Non-intervention to the aerial way exists, which performs two functions, namely:
a) The larynx non-instrumentalization permits to maintain spontaneous pulmonary ventilation whether in prone or ventral decubitus.
b) As a phonation organ, the non-instrumentalization of the larynx facilitates the permanent communication between the anesthetist and the surgeon. In this way, the patient is capable to accurately monitor any stimulus being potentially noxious on a nervous root and forces the surgeon to modify the surgical approximation in the operating zone (“MNCC”). In turn, the verbal stimulus permits to perform a continuous evaluation of functional tests (“ECPFN”).

The conscious and participative sedation condition exceptionally prevents unsuitable conditions of over-sedation or deep sedation that could generate ventilation, hemodynamic problems and eventual neurological injuries upon the interrupted control of communication with the patient.

The sedation level can be flexibly varied according to the surgical requirement and the possible and adequate clinical evaluation (“MNCC” and “ECPFN”).

Substantial hemodynamic and ventilatory stability are generated by an optimum seudo-analgesic condition when performing these minimal spinal column invasive techniques.

The eventual hemodynamic alterations such as hypotension and bradycardia are corrected by means of drugs usually used in any anesthetic technique, such atropine and ephedrine in customary doses. Arterial hypotension has been defined as a 30% reduction of the tensional figures evaluated in the immediate pre-operatory procedure. Bradycardia has been defined as a cardiac frequency under 50 heart throbs per minute.

The commencement and completion of conscious sedation are shorter than those produced by general anesthesia. The intra-operatory and immediate post-operatory times are shorter than those produced by general anesthesia. The clinical discharge of these patients can be potentially precocious (before eight hours) in perfect condition.

The cost of inputs resulting from this conscious sedation method can be smaller than that resulting from general anesthesia.

This method directly benefits the surgery patients, which include spine endoscopic surgeries and other applications both inside and outside the operating theater, but it is more evident in patients in advanced age groups having associated pathologies, due to a suitable safety level and scant adverse events and damages attributable to the patient position.

The possibility to evaluate the intra-operatory and neurologic situation of the patient In real time, by applying this conscious sedation method improves the performance expectations and efficiency, both in respect to sedation and surgical technique by reducing the patient's morbidity as compared with inadequate sedation techniques or conditions (over-sedation or excessive wakefulness).

### Advantages of Conscious Sedation

Excellent control of the conscience degree, anxiety and pain during the surgery.
Great stability of the patient's physiological parameters (blood pressure, pulse, blood oxygenation and awareness degree).
This type of anesthesia permits to operate older age patients, even suffering stabilized associated pathologies (cardiopathy, blood hypertension, diabetes and those of a respiratory, renal and neurological nature), as well as patients being prostrated due to their own spine pathologies.
The conscious sedation condition permits—at all times—that the patient talks with the surgeon and the anesthetist. Moreover, the patient can see, understand and collaborate through a video monitor during his/her surgery.
The above permits the patient-surgeon communication during the operation and also to locate the real and precise pain source, without damaging him/her.
The drugs being used permit a fast recovery.
The method can be translated into saving of inputs and bed-hours.

### DETAILED DESCRIPTION OF THE INVENTION

Below is a description of an example of use of the new invention in surgeries:

### 1. Patient Position in the Surgery

a. Lumbar and Dorsal Spine Column Surgery.

The most used position in this surgery is prone decubitus with 90° bent lower extremities (hip or femur over the trunk and legs over the thighs); to this effect a Relton & Hall type table is used. Due care should be taken at all times about the compression or discomfort of the different supporting points of the patient body. The head can be in a neutral position or naturally turned into both sides. The trunk is placed slightly bent for a better exposure of the dorsal spine. The thorax and abdomen should not be compressed, which permits the patient to comfortably breathe. Non-compression of the abdomen avoids the vena cava thereby reducing the venous pressure at the venous plexus level, which covers the nervous and osseous structure of the spinal column (epidural venous plexus), and remarkably improving the vision of the surgical field (endoscopic surgery) causing its less bleeding.

b. Cervical Spinal Column Surgery.

The position for a backward approach to the cervical spine of a patient undergoing cervical spine surgery through endoscopic or minimal invasive technique is prone decubitus fully extended, thus protecting the supporting points and looking for the patient comfort. The cervical region should show a slight bending in order to best expose the posterior cervical zone and the head position should be in the center and looking downwards.

To the effects of the front approach to the cervical spinal column, the patient is placed in supine decubitus position, the knees being slightly bent (a pad being placed in the popliteal cavals) and protection to the heels. The moderate extension of the cervical spine is desirable as well as a deviation of the head at the side lateral counter side of the surgical approach.

In both cases an approximately 5 to 10 cm high pad should be placed under the thorax in a slight Fowler position (15 to 20°). In addition, the head is fixed in a central position at the level of the forehead or occipital region, as it corresponds, in order to achieve immobility of the cervical region during the surgery.

### 2. Monitoring of the Patient

The necessary monitoring in a minimally invasive endoscopic surgery of the spinal column comprises the following:

a. Continuous Electrocardiogram with 3 or 4 Derivations.

It permits to see the cardiac frequency and hemodynamic events that affect the electric activity or the heart function.

b. Measurement of Oxygen Arterial Saturation.

It is performed using a device (pulse oximeter) that permits the continuous analysis of oxygen level variations in the blood.

c. Measurement of Non-Invasive Arterial Pressure.

A digital automatic system is used. This measurement permits to evaluate the variations of arterial pressure during the surgical procedure.

d. Capnography.

The continuous measurement of exhaled CO₂ and the continuous visualization of the capnographic curve permit to jointly monitor the pulmonary ventilation and its eventual depression or alteration degree. In an optimum manner, a special device is used (bi-functional nosepiece) that permits to administer oxygen and at the same time the capture of the exhaled CO₂ for analysis in an ad hoc monitor. It is not customary in the course of sedation to monitor the CO₂. In this case, the sedation for spinal column in endoscopic surgery is undoubtedly a great contribution in terms of the required monitoring action, moreover when we have prone decubitus positions with fluctuating conscience condition which could cause potentially serious ventilatory and hemodynamic alterations.

e. Continuous Conscious Neurological Monitoring (MNCC) and Continuous Evaluation of Neurological Functional Tests (EPCFN).

The above two concepts arise from the clinical valuation during the intra-operatory process of patients under spine endoscopic surgery under conscious sedation. The fact of being able to perform said evaluations both by the surgeon and the anesthetist (verbally and directly with the patient) constitute an essential fact of the method, due to the neurological protection produced at all times and to the 100% elimination of potential neurological damages. Usually, a slight to moderate pain can exist, as well as paresthesia and some type of effector response. This communication in real time permits to minimize, avoid or eliminate the potential direct aggression to the nervous roots, which are not completely visible in the endoscopic surgical field, and that would be damaged by the instruments and technological elements used.

### “MNCC”:

This evolves through the permanent communication among the patient, the anesthetist and the surgeon in the course of the surgery under conscious sedation. Said continuous monitoring takes place by means of simple and definite questions which are specific about the type and location of the pain, with emphasis on the neuropathic origin pains (dysesthesias, paresthesia) whether caused by mechanic, electric, of radiofrequency, laser or other stimuli.

### “EPFNC”:

Through the permanent communication among patient, anesthetist and surgeon, the patient is ordered under conscious sedation to move some segment of his/her body, to define and evaluate the location of some pain or sensitive or motor deficit. In this way the advance of the surgical instrumental advance can be corrected and avoid the potential damage.

f. Ramsay Sedation Scale

Level 1: Agitated, anxious or restless patient.

Level 2: Cooperative, oriented, calm patient.

Level 3: Sleeping patient who answers verbal orders.

Level 4: Sleeping patient, briefly answers to the light and sound.

Level 5: Patient is sleeping and only responds to pain.

Level 6: Patient does not give any answer.

### 3. Oxygen Therapy

Oxygen (O₂) administration in a fixed 100% quantity of 4 liters per minute through a special device (bifunctional nose-piece).

### 4. Infusion Electronic Pumps

Infusion electronic pumps are used for administrating the necessary specifics for achieving sedation (dexmedetomidine, phentanil and propophol) and some lidocaine or other brief action opiate.

### 4. Venous Accesses

Teflon No 20 or 18 is used, which permits fluids administration (crystalloids, colloids or blood derivatives) and the method drugs for conscious sedation.

During the drugs administration, two in line by-pass valves are used, which are related to the fluids lines to be administered, all of the foregoing being united to feflon. Administration through one of them then proceeds from the infusion pump (“TCI”). By means of the other by-pass valve dexmedetomidine and phentanil are jointly supplied, both coming from a dual-channel volumetric pump.

We could ideally count with a three-channel infusion electronic pump with 3 lumen fluids line. In this way the drugs administration could be simplified both sequentially and simultaneously in order that the drugs finally enter in the circulatory system and produce the desired clinical effects.

### 6. Drugs Used in the Anesthetic Method

The drugs being involved in this conscious sedation anesthetic method in spinal column endoscopic surgeries are:

Dexmedetomidine hydrochloride

Propophol

Phentanil

Some lidocaine or other brief action opiate

### 7. Other Specific Drugs

Analgesics, non-steroidal anti-inflammatory drugs

Corticoids

Gastric mucosa protecting drugs

Antibiotics

### 8. Infiltration Local Anesthesia

Following the sedation consolidation stage and prior to the surgery, local infiltration anesthesia is performed using approximately 2% and 1% lidocaine or other brief action opiates. These could be eventually mixed and associated to other local anesthetics offering longer action duration or others that provide a good clinical effect and safety. A vasoconstrictor and bicarbonate associated to local anesthetics are possible to use in order to obtain a shorter latency and less bleeding in the surgical accesses.

The infiltration should be performed in a meticulous and careful manner. Absolute non-sensibility of both superficial structures (dermis) and deep structures (muscles, facia, periosteum, etc.) should exist. The use of additional doses of local anesthetics could be necessary for the non-sensitization of the above structures.

The maximum recommended doses should not exceed the use of each local anesthetic drug.

### Drugs—Composition and Method—Sequence

### Conscious Sedation Scheme

A sequence has been proposed regarding the administration of these drugs (dexmedetomidine, propophol and phentinil) and some lidocaine or other brief action opiate which provide a great clinical safety in low doses and when being associated rationally and adapted to the surgical needs and the possibility of an exhaustive clinical monitoring of the intra-operatory procedures offer exceptional benefits to patients being submitted to diverse surgery techniques, including spinal column endoscopic surgery and other applications.

A least four stages are identified in this conscious sedation scheme, namely:

### Stage 1: Installation of Sedation

The full monitoring procedure already described is followed, i.e. the endovenous tract is placed and the customary endovenous solution.

This stage commences before the anesthesia infiltration. Once the patient has been monitored and a permeable venous tract has been installed, the simultaneous administration commences of dexmedetomidine in one dose of 0.5 mg/kg/weight and propophol in doses fluctuating between 0.3 and 1.2 ug/kg/hour during 10 minutes.

In the lapse of this time, the clinical patient response to these drugs is evaluated; the propophol infusion rate could vary with a view to achieving an adequate sedation condition (somnolence, anxiolysis). To this effect the Ramsay Sedation Levels Scale is used and Levels 1 and 3 are maintained. Additionally, it is necessary to observe the clinical response of the patient to dexmedetomidine (anti-adrenergic effects), that can cause a drop of the blood pressure and of the cardiac frequency, further to sedative and analgesic effects inherent in this drug (watched sedation), fifteen minutes before local anesthesia and after commencement of the surgery.

### Stage 2: Analgesia Reinforcement

After completion of Stage 1, the administration of a bolus of phentanil in doses of 1 ug/kg/weight to be passed in 5 minutes.

By way of additional safety measure, upon commencement of this stage the propophol infusion range is reduce within a range of 0.2 to 0.5 micrograms per kg per hour in respect to the previously established level. Also some lidocaine or other short action opiate can be administered.

### Stage 3: Consolidation of the Sedation

Upon completion of Stage 2 a clinical evaluation of the sedation level prior to infiltration with local anesthetics is performed, subsequently to the commencement of the surgery. It is customary to establish adequate clinical conditions for starting the surgery, whose stimuli are often moderate (skin or dermis infiltration) and then more intensive when the surgical accesses using instruments occurs.

During this stage “MNCC” and “ECPFN” are exhaustively performed.

### Stage 4. Maintenance of Sedation

During this stage the purpose is to vary the propophol dose in order to obtain the best possible sedation condition and the best stability of hemodynamic and ventilatory variables. Also some lidocaine or other brief action opiate is used.

In addition, and exceptionally a reinforcement with phentonil bolus of 0.5 ug/kg/weight is used, to be passed in 5 minutes to strengthen the analgesia in this period, if necessary. This dose would be repeated in the course of the surgery, as necessary.

In this stage, “MNCC” and “ECPFN” are regulatory and their implementation should be strictly complied with. The patient should be able to verbally respond when faced by painful stimuli and specially the stimulus produced in nervous roots (thermal, electric and mechanic) and the consequent motor response and the accurate location of paresthesia provoked in the involved nervous root.

1. A method and doses for the administration of drugs, CHARACTERIZED in that for obtaining conscious sedation using bio-backfeed, in communication with the patient him/herself for use in surgeries, including spine column and other surgeries and applications inside and outside the operating theater, wherein the fact of having a conscious patient permits greater safety and/or reduction of risks.

2. A method and doses for conscious sedation in surgeries, CHARACTERIZED in that by including endoscopic spine column surgeries and other surgeries and applications inside and outside the operating theater and using low doses of dexmedetomidine, phentanil and propophol and some lidocaine or other short action opiate, the risks of injuries is reduced and/or safety is increased.

3. Method and doses CHARACTERIZED in that for conscious sedation in surgeries, including spinal column endoscopic and other surgeries and applications inside and outside the operating theater, the risk of injuries is reduced because the sedation method for lumbar and dorsal spine surgeries comprises the following stages:
a) Installation of sedation. The full monitoring procedure is followed; the endovenous tract is placed as well as the customary endovenous solution. In this stage the infiltration commences prior to anesthesia, once the patient is monitored and a permeable venous tract has been installed, simultaneous administration of dexmedetomidine and propophol commences plus some lidocaine or other of brief action opiate in doses of 0.5 .mg/kg/weight with propophol in doses fluctuating between 0.3 and 1.2 ug/kg/hour during 10 minutes; in the course of this time the patient clinical response to the infusion of these drugs starts; the infusion rate of propophol can be varied in order to achieve an adequate sedation condition (somnolescence, anxiolysis). To this effect the Ramsay Sedation Levels Scale is used and 2 and 3 levels are maintained; it is additionally necessary to observe the clinical response of the patient to dexmedetomidine (anti-adrenergic effects) which could provoke a reduction of the blood pressure and the cardiac frequency, further to sedative and analgesic effects inherent in this drug (watched sedation), fifteen minutes prior the infiltration of local anesthesia after the surgery commences.
b) Upon completion of Stage a, the administration of a phentanil bolus in 1 ug/kg/weight starts, to be passed in 5 minutes; by way of additional safety measure upon commencement of this stage, the infusion rate of propophol is reduced by a range of 0.2 to 0.5 mg per kg per hour in respect to the previously established level.
c) Upon completion of Stage b, a clinical evaluation is performed of the sedation level prior to infiltration with local anesthetics and after surgery commences. The adequate clinical conditions are established for starting the surgery, whose stimuli are often moderate (skin infiltration) and then more intensive by instrumenting the surgical accesses. During this stage “MNCC” and “ECPFN” are performed in an exhaustive form, and
d) Finally, maintenance of sedation should be made by varying the propophol doses with a view to obtaining the best possible sedation condition and the best stability of hemodynamic and ventilatory variables. In addition, exceptionally a reinforcement used. consisting of a bolus of phentanil with a 0.5 ug/kg/weight, to be passed in 5 minutes in order to strengthen the analgesia in this period, if necessary. This dose can be repeated in the course of the surgery, if necessary.

4. Method and dose for conscious sedation in surgeries, including spinal column endoscopic surgery and other applications inside and outside the operating theater, which reduce the risks of injuries because the sedation method for lumbar and dorsal spinal column comprises the following stages:
a) Installation of Sedation. The whole monitoring procedure is followed;
endovenous tract is placed as well as the customary endovenous solution. In this stage and prior to the anesthesia infiltration. Once the patient has been monitored. And has a permeable a venous tract duly installed the simultaneous administration of dexmedetomidine and propophol; the clinical response of the patient to the infusion of these drugs is evaluated commences and the moderate rate of propophol infusion can vary in order to achieve an adequate sedation condition (somnolescence, anxiolysis); in addition it is necessary to observe the patient clinical response to dexmedetomidine (anti-adrenergic effects) that could provoke a reduction of the blood pressure and of the cardiac frequency, further to sedative and analgesic inherent in this drug (watched sedation).
b) Upon completion of the Stage (a), administration of phentanil starts; by way of additional safety upon commencement of this stage, the infusion rate of propophol is reduced in respect to the previously established level.
c) Once Stage (b) is completed, a clinical evaluation of the sedation level prior to the infiltration with local anesthetics and the subsequent commencement of the surgery;
the adequate clinical conditions are established for starting the surgery, whose stimuli are usually moderate (skin infiltration) and then more intensive upon instrumenting the surgical accesses. During this Stage, “MNCC” and “ECPFN” are performed in an exhaustive form, and
d) Finally, maintenance of sedation should be made and the propophol dose would be varied for obtaining the possible sedation conditions and the best stability of hemodynamic and ventilator variables. In addition, and exceptionally, a reinforcement with bolus of phentanil is applied in order to strengthening the analgesia in this period if necessary: the latter procedure can be repeated in the course of the surgery, as necessary.
